(19) United States
(12) Patent Application Publication
(10) Pub. No.: US 2020/0353223 A1
(43) Pub. Date: Nov. 12, 2020

(54) FLAGELLA BALLOON CATHETER AND WIRE

(71) Applicant: THE JOHNS HOPKINS UNIVERSITY, Baltimore, MD (US)

(72) Inventors: Andrew Schulick, Bethesda, MD (US); Devika Singh, Bethesda, MD (US)

(21) Appl. No.: 16/762,202

(22) PCT Filed: Nov. 6, 2018
(86) PCT No.: PCT/US2018/059363

§ 371 Date: May 7, 2020

Related U.S. Application Data
(60) Provisional application No. 62/582,404, filed on Nov. 7, 2017.

(51) Int. Cl. A61M 25/09; A61M 25/01

(52) U.S. Cl. CPC A61M 25/09; A61M 25/0147; A61M 2025/09141; A61M 2025/09175; A61M 2025/09008

(57) ABSTRACT

A flagellated, back-curved guidewire component of the present invention facilitates passage of a balloon catheter through the vasculature. The guidewire may be used in conjunction with commercially available devices. Alternately, the guidewire may come integrated with a balloon catheter, such as a flagellated balloon embolectomy catheter or a flagellated balloon resuscitation catheter. The guidewire can include multiple flexible flagellate disposed at a distal end of an elongate wire configured for passing through the vasculature. The guidewire is configured to self-center in the vasculature.

### CROSS REFERENCE TO RELATED APPLICATION

This application claims the benefit of U.S. Provisional Patent Application No. 62/582,404 filed on Nov. 7, 2017, which is incorporated by reference, herein, in its entirety.

### FIELD OF THE INVENTION

The present invention relates generally to medical devices. More particularly, the present invention relates to a flagellated wire component for use with a balloon catheter.

### BACKGROUND OF THE INVENTION

Placement of balloon catheters through arteries (and sometimes veins) without imaging guidance is a common requirement in open vascular surgery, and in trauma surgery. Resuscitation aortic balloon catheters are gaining acceptance as adjuncts to augment blood pressure during resuscitation of victims of torso trauma not amenable to pressure tamponade. Balloons are placed percutaneously from the femoral arteries, advanced into the aorta and inflated. By temporarily blocking blood flow to the lower half of the body, blood flow is augmented to the more proximal vital organs (brain, heart, lungs, liver, kidney, gut, etc). FIGS. 1A and 1B illustrate side views of blind placement of occlusion balloons, according to the prior art. Placement of these balloons is generally performed blindly, with or without the aid of a guidewire, as illustrated in FIG. 1A. As noted above, blind attempts at placement may be unsuccessful, or even worse, the resuscitation balloon may be misplaced (with or without a guide wire), resulting in additional damage upon inflation, as illustrated in FIG. 1B. Even newer versions of resuscitation balloons used in trauma are still subject to the same drawbacks, including inability to place, and misplacement.

Restoring flow to peripheral arteries occluded as a consequence of atrial fibrillation-mediated cardioemboli, is one of the most common emergency operations in vascular surgery. The procedure involves cutting down onto the artery, opening the vessel, and passing inflatable balloon embolectomy catheters proximally and distally to clear out the occlusion, as illustrated in FIGS. 2A-2C. FIGS. 2A-2C illustrate steps for restoring blood flow to peripheral arteries occluded as a consequence of atrial fibrillation-mediated cardioemboli, as is known in the art.

Inability to pass the catheter through the clot is a common problem, as illustrated in FIG. 3. FIG. 3 illustrates a side view of a catheter that cannot be advanced past a clot, as is known in the prior art. The catheter can get hung up at any area of irregularity, including small plaques or side branches. The problem can be difficult to troubleshoot since the catheter is being passed blindly (without image guidance). The result is inability to restore blood flow. The consequence is conversion of a simple embolectomy procedure (30 minutes) into a hybrid endovascular/open procedure (which adds significant complexity, and costs valuable time), or more commonly conversion into open bypass procedure (2 hours) to restore flow.

Accordingly, there is a need in the art for a flagellated wire component for use with a balloon catheter.

### SUMMARY OF THE INVENTION

The foregoing needs are met, to a great extent, by the present invention which provides a device for passage through vasculature. The device includes an elongate wire body. The elongate wire body has a distal end and a proximal end configured to pass through the vasculature. The device also includes a leading end comprising at least two flagella. The flagella are curved back toward the proximal end of the elongate wire body, and the leading end is coupled to a distal end of the elongate wire body.

In accordance with an aspect of the present invention, the elongate wire body and the leading end are formed from a biocompatible metal. The device can include a balloon. The balloon is integrated into the elongate wire body. The balloon takes the form of a balloon catheter having a lumen. The leading end can pass through the lumen. The device includes a guidewire introducer. Additionally, the device can include a guidewire sheath. The at least three flagella are formed from a shape memory metal. The at least two flagella are configured to exert a radial force on a wall of a vessel, such that the elongate wire body remains centered in the vessel. The flagella are configured to return to a position of being curved back toward the proximal end of the elongate wire body. The guidewire can include a hydrophilic coating, an anti-thrombogenic coating, a hydrophobic coating, or friction reducing coating.

In accordance with another aspect of the present invention, a device for passage through vasculature includes an elongate wire body having a distal end and a proximal end configured to pass through the vasculature. The device includes a leading end having at least two flagella. The at least two flagella are curved back toward the proximal end of the elongate wire body, and the leading end is coupled to a distal end of the elongate wire body. The device also includes a balloon disposed proximal to the leading end. The balloon is in an uninflated configuration prior to insertion of the device through the vasculature, and the balloon is configured to be inflated. The balloon includes an insufflation lumen to allow for inflation of the balloon.

In accordance with yet another aspect of the present invention, the elongate wire body and the leading end are formed from a biocompatible metal. The at least two flagella are formed from a shape memory metal. The at least two flagella are configured to exert a radial force on a wall of a vessel, such that the elongate wire body remains centered in the vessel. The flagella are configured to return to a position of being curved back toward the proximal end of the elongate wire body. The guidewire can include a hydrophilic coating, an anti-thrombogenic coating, a hydrophobic coating, or friction reducing coating.

In accordance with still another aspect of the present invention, a device for passage through vasculature includes an elongate wire body having a distal end and a proximal end configured to pass through the vasculature. The device includes a leading end having at least two flagella. The at least two flagella are curved back toward the proximal end of the elongate wire body, and the leading end is coupled to a distal end of the elongate wire body. The device also includes a delivery catheter defining an elongate lumen extending therethrough, wherein the lumen is configured to receive the elongate wire body and its leading end.

In accordance with even another aspect of the present invention, the delivery catheter includes a balloon. The balloon is in an uninflated configuration prior to insertion of the device through the vasculature, and the balloon is configured to be inflated. The delivery catheter includes an insufflation lumen to allow for inflation of the balloon. The at least two flagella are positioned in the delivery catheter in an extended position.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIGS. 1A and 1B illustrate side views of blind placement of occlusion balloons, according to the prior art.

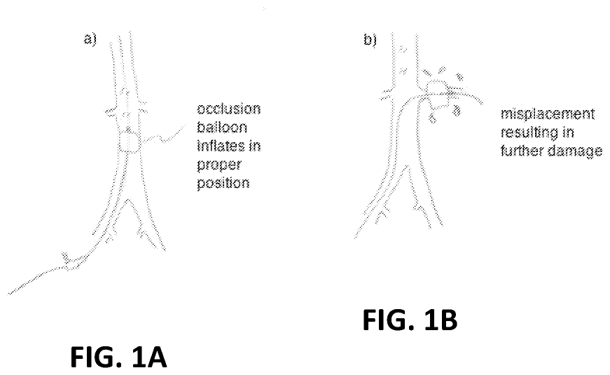

FIGS. 2A-2C illustrate steps for restoring blood flow to peripheral arteries occluded as a consequence of atrial fibrillation-mediated cardioemboli, as is known in the art.

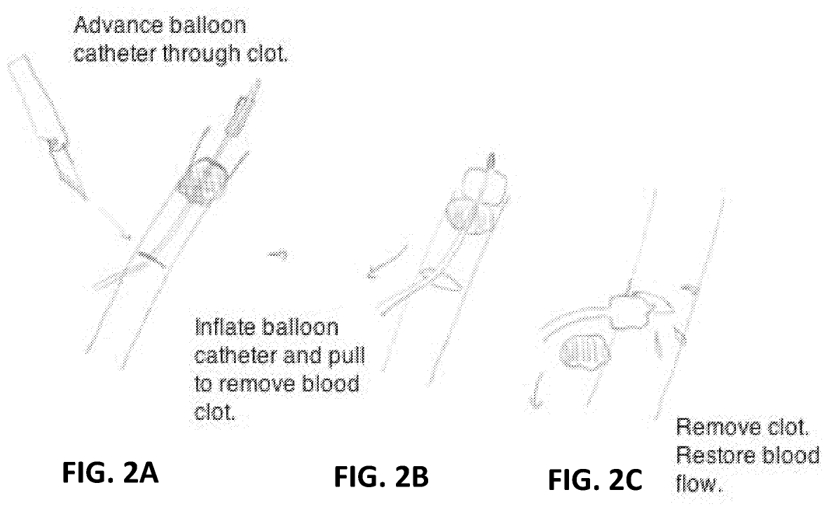

FIG. 3 illustrates a side view of a catheter that cannot be advanced past a clot, as is known in the prior art.

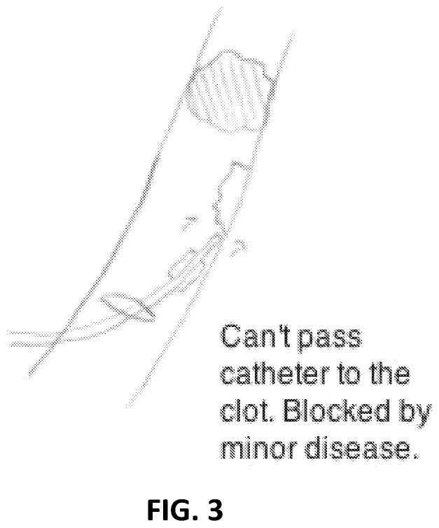

FIGS. 4A-4C illustrate perspective and end views of guidewires with flagella, according to an embodiment of the present invention.

FIG. 5 illustrates a side view of a guidewire with flagella coupled to a resuscitation balloon and disposed in a vessel, according to an embodiment of the present invention.

FIG. 6 illustrates a side view of a guidewire with flagella, according to an embodiment of the present invention, moving through a diseased blood vessel.

FIGS. 7A-7C illustrate side views of a guidewire of the present invention being used in conjunction with an embolectomy balloon catheter to extract a clot from an irregular blood vessel.

FIG. 8A illustrates perspective views of a guidewire and introducer, and FIG. 8B illustrates a sheath, both according to an embodiment of the present invention.

FIGS. 9A and 9B illustrate perspective views of a guidewire with integrated balloon, according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention is directed to a flagellated, back-curved guidewire component, which facilitates passage of a balloon catheter through the vasculature. The guidewire may be used in conjunction with commercially available devices. Alternately, the guidewire may come integrated with a balloon catheter, such as a flagellated balloon embolectomy catheter or a flagellated balloon resuscitation catheter. The guidewire can include multiple flexible flagella disposed at a distal end of an elongate wire configured for passing through the vasculature. The guidewire is configured to self-center in the vasculature.

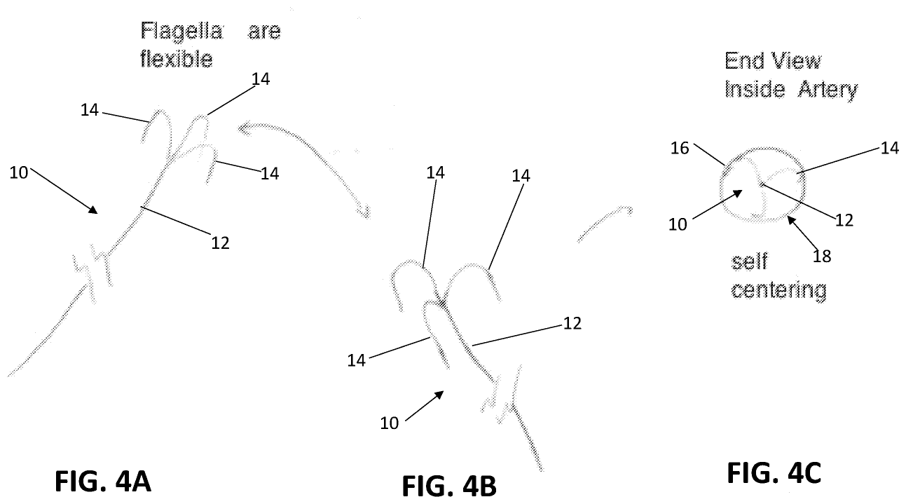

FIGS. 4A-4C illustrate perspective and end views of guidewires with flagella, according to an embodiment of the present invention. As illustrated in FIGS. 4A-4C, the guidewire 10 includes is a trifurcated, flexible, and backwards bending tip 12. Each of the furcations of the tip of the guidewire is referred to as a flagella 14. The flagella 14 apply a light radial force on the wall 16 of the vessel 18, as illustrated in FIG. 4C. The light radial force applied by the flagella 14 keeps the main trunk of the wire centered within a lumen of the vessel. The flagella 14 can be compressed to fit within a delivery catheter or vessels of various sizes. The flagella 14 are also configured to expand when more space is provided. The guidewire 10 also includes an elongate body 20. The guidewire 10 and flagella can be formed from any suitable biocompatible material such as a metal, metal alloy, or a shape memory metal alloy. The guidewire 10 can include a coating, such as a hydrophilic coating, anti-thrombogenic coating, hydrophobic coating, or friction reducing coating. The tension or resistance in the flagella 14 to allow the guidewire to remain centered is generated by the material chosen for forming the flagella 14. Therefore, different materials could be used to generate different resistance based on the vasculature or use of the guidewire with flagella.

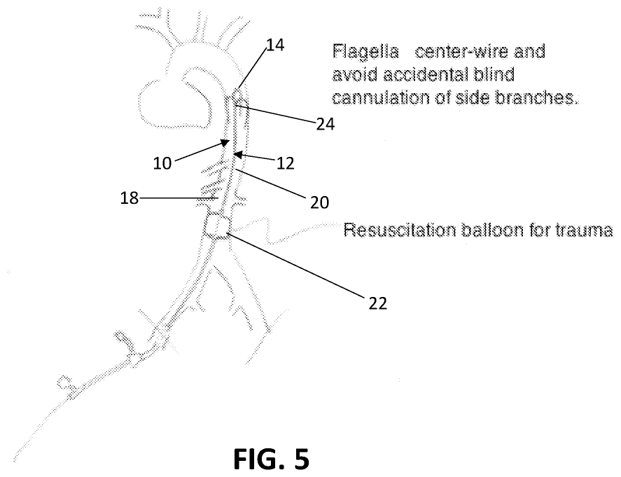

FIG. 5 illustrates a side view of a guidewire with flagella coupled to a resuscitation balloon and disposed in a vessel, according to an embodiment of the present invention. As illustrated in FIG. 5 the guidewire 10 extends distal to the resuscitation balloon 22. The elongate body 20 of the guidewire 10 extends through the vessel 18. Flagella 14 are disposed at a distal end 24 of the guidewire 10. The flagella 14 center the guidewire 10 within the vessel 18. The radial force applied by the flagella 14 serve to avoid accidental blind cannulation of side branches of the vasculature. The guidewire 10 with flagella 14 also allows the resuscitation balloon to pass across arterial branches. The resuscitation balloon 22 follows through the vessel 18 proximal to the guidewire 10. The guidewire 10 and the resuscitation balloon 22 can be coupled in any way known to or conceivable to one of skill in the art, including but not limited to, the guidewire being disposed through a lumen of a catheter associated with the resuscitation balloon.

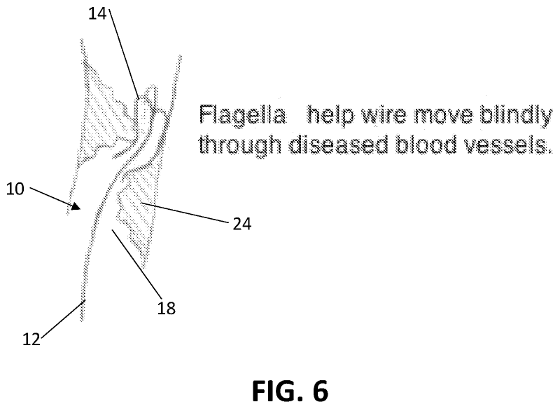

FIG. 6 illustrates a side view of a guidewire with flagella, according to an embodiment of the present invention, moving through a diseased blood vessel. As illustrated in FIG. 6, the vessel 18 includes diseased areas 24. The diseased areas 24 can take the form of plaques, clots, other blockages in the artery, or any other source of luminal irregularity. As the guidewire 10 extends through the vessel 18, the flagella help the wire to move blindly through the vessel 18, by exerting radial force on the diseased areas. The radial force centers the body of the guidewire 10 and allows it to move past the diseased areas 24.

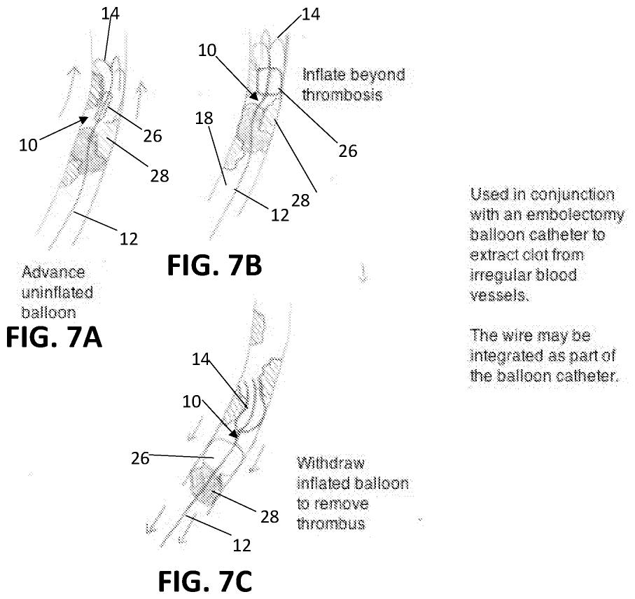

FIGS. 7A-7C illustrate side views of a guidewire of the present invention being used in conjunction with an embolectomy balloon catheter to extract a clot from an irregular blood vessel. As illustrated in FIG. 7A-7C, a guidewire 10 according to an embodiment of the present invention is coupled to an embolectomy balloon catheter 26. In FIG. 7A, the embolectomy balloon catheter 26 is uninflated and is being advanced through the vessel 18, proximal to the flagella 14 of the guidewire 10. As described above, the flagella 14 exert a radial force on the vessel in order to center the guidewire 10 during advancement. This allows the guidewire 10 and coupled embolectomy balloon catheter 26 to move past the clots 28 in the vessel. The guidewire 10 may be integrated into the balloon catheter 26 or removably disposed through a lumen of the embolectomy balloon catheter 26. Any other suitable way of delivering the balloon catheter 26 in conjunction with the guidewire 10 known to or conceivable to one of skill in the art can also be used. As illustrated in FIG. 7B, the embolectomy balloon 28 is inflated just beyond the thrombosis. As illustrated in FIG. 7C, the inflated embolectomy balloon is withdrawn back through the vessel 18 in order to remove the thrombus. Further with respect to FIG. 7C, when the guidewire 10 is withdrawn, the flagella 14 invert in order to allow for easier removal of the guidewire 10.

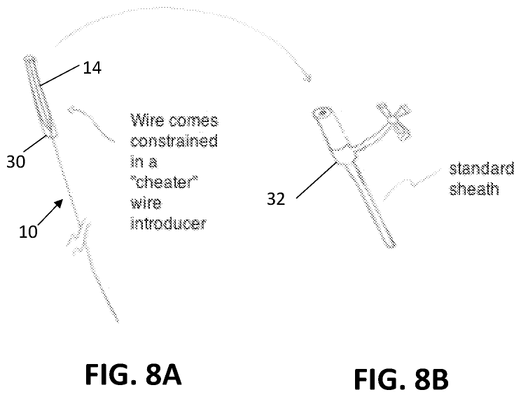

FIG. 8A illustrates perspective views of a guidewire and introducer, and FIG. 8B illustrates a sheath, both according to an embodiment of the present invention. As illustrated in FIG. 8A, the guidewire 10 can come constrained in a “cheater” wire introducer 30. The wire introducer 30 holds the flagella 14 in an extended position. Insertion can be achieved using the sheath 32 illustrated in FIG. 8B. The guidewire 10 is designed to be used with commercially available sheaths, and is introduced using a simple guidewire introducer.

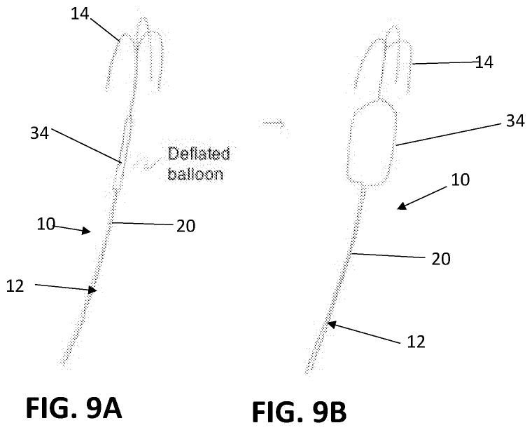

FIGS. 9A and 9B illustrate perspective views of a guidewire with integrated balloon, according to an embodiment of the present invention. As illustrated in FIGS. 9A and 9B, the guidewire 10 can include an integrated balloon 34 positioned proximal to the flagella 14 on the body 20 of the guidewire 10. The balloon 34 is illustrated in a deflated configuration in FIG. 9A and an inflated configuration in FIG. 9B. The guidewire 10 can include an insufflation lumen for inflation of the balloon 34. Alternately, the balloon can be inflated by the introducer, sheath, associated catheter, or any other means known to or conceivable to one of skill in the art. The flagellated wire may be a stand-alone device, or in separate embodiments it may be integrated onto balloon catheters, simplifying use, especially in emergent trauma situations.

The many features and advantages of the invention are apparent from the detailed specification, and thus, it is intended by the appended claims to cover all such features and advantages of the invention which fall within the true spirit and scope of the invention.

Further, since numerous modifications and variations will readily occur to those skilled in the art, it is not desired to limit the invention to the exact construction and operation illustrated and described, and accordingly, all suitable modifications and equivalents may be resorted to, falling within the scope of the invention. While exemplary embodiments are provided herein, these examples are not meant to be considered limiting. The examples are provided merely as a way to illustrate the present invention. Any suitable implementation of the present invention known to or conceivable by one of skill in the art could also be used.

1. A device for passage through vasculature comprising:
an elongate wire body having a distal end and a proximal end configured to pass through the vasculature; and
a leading end comprising at least two flagella, wherein the flagella are curved back toward the proximal end of the elongate wire body, and wherein the leading end is coupled to a distal end of the elongate wire body.

2. The device of claim 1 further comprising the elongate wire body and the leading end being formed from a biocompatible metal.

3. The device of claim 1 further comprising a balloon.

4. The device of claim 3 wherein the balloon is integrated into the elongate wire body.

5. The device of claim 3 wherein the balloon takes the form of a balloon catheter comprising a lumen wherein the leading end can pass through the lumen.

6. The device of claim 1 further comprising a guidewire introducer.

7. The device of claim 1 further comprising a guidewire sheath.

8. The device of claim 1 further comprising the at least two flagella being formed from a shape memory metal.

9. The device of claim 1 wherein the at least two flagella are configured to exert a radial force on a wall of a vessel, such that the elongate wire body remains centered in the vessel.

10. The device of claim 1 wherein the at least two flagella are configured to return to a position of being curved back toward the proximal end of the elongate wire body.

11. The device of claim 1 wherein the guidewire comprises one selected from a group consisting of a hydrophilic coating, anti-thrombogenic coating, hydrophobic coating, or friction reducing coating.

12. A device for passage through vasculature comprising:
an elongate wire body having a distal end and a proximal end configured to pass through the vasculature;
a leading end comprising at least two flagella, wherein the flagella are curved back toward the proximal end of the elongate wire body, and wherein the leading end is coupled to a distal end of the elongate wire body; and,
a balloon disposed proximal to the leading end, wherein the balloon is in an uninflated configuration prior to insertion of the device through the vasculature, wherein the balloon is configured to be inflated, and wherein the balloon includes an insufflation lumen to allow for inflation of the balloon.

13. The device of claim 12 further comprising the elongate wire body and the leading end being formed from a biocompatible metal.

14. The device of claim 12 further comprising the at least two flagella being formed from a shape memory metal.

15. The device of claim 12 wherein the at least two flagella are configured to exert a radial force on a wall of a vessel, such that the elongate wire body remains centered in the vessel.

16. The device of claim 12 wherein the at least two flagella are configured to return to a position of being curved back toward the proximal end of the elongate wire body.

17. The device of claim 12 wherein the guidewire comprises one selected from a group consisting of a hydrophilic coating, anti-thrombogenic coating, hydrophobic coating, or friction reducing coating.

18. A device for passage through vasculature comprising:
an elongate wire body having a distal end and a proximal end configured to pass through the vasculature;
a leading end comprising at least two flagella, wherein the flagella are curved back toward the proximal end of the elongate wire body, and wherein the leading end is coupled to a distal end of the elongate wire body; and,
a delivery catheter defining an elongate lumen extending therethrough, wherein the lumen is configured to receive the elongate wire body and its leading end.

19. The device of claim 18 wherein the delivery catheter further comprises a balloon, wherein the balloon is in an uninflated configuration prior to insertion of the device through the vasculature, wherein the balloon is configured to be inflated, and wherein the delivery catheter includes an insufflation lumen to allow for inflation of the balloon.

20. The device of claim 18 wherein the at least two flagella are positioned in the delivery catheter in an extended position.
